(19) United States
(12) Patent Application Publication
(10) Pub. No.: US 2003/0104090 A1
(43) Pub. Date: Jun. 5, 2003

(54) Supplements containing annatto extracts and carotenoids and methods for using the same

(76) Inventors: Pedro E. Levy, Longmeadow, MA (US); Luis W. Levy, Quito (EC)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/309,732

(22) Filed: Dec. 4, 2002

Related U.S. Application Data
(63) Continuation-in-part of application No. 09/565,957, filed on May 5, 2000.
(60) Provisional application No. 60/336,637, filed on Dec. 4, 2001.

(51) Int. Cl. A61K 35/76

(52) U.S. Cl. 424/776; 424/195.17; 424/750; 424/757; 424/777; 424/764; 424/780

(57) ABSTRACT

Methods are provided for eliciting a beneficial health effect in a human or non-human animal which include adding at least one bixin compound or an extract of the annatto (Bixa orellana) seed to an animal feed, food product or nutritional supplement in an amount effective to increase the concentration of bixin in the blood supply of the animal when the food or supplement is consumed at a recommended daily dosage (unit dosage) for a sufficient number of days. A method of eliciting a therapeutic health effect in a human or non-human animal by adding an effective amount of at least one bixin compound to a pharmaceutical compound is also provided. The beneficial or therapeutic health effect may be further enhanced by the addition of at least one carotenoid compound or carotenoid-containing extract to the feed, food product, nutritional supplement, or pharmaceutical composition. These methods are particularly suited to companion animals, such as cats and dogs, and to farmed animals, such as layer hens, broiler chickens, and pigs.

### CROSS-REFERENCE TO RELATED APPLICATIONS

This application claims the benefit of U.S. Provisional Application No. 60/336,637, filed Dec. 4, 2001, and is a continuation-in-part of co-pending U.S. patent application Ser. No. 09/565,957, filed May 5, 2000.

### BACKGROUND OF THE INVENTION

Annatto extracts are obtained from the seeds of the tropical tree Bixa orellana. Annatto seed extracts contain bixin compounds, the isoprenoid geranylgeraniol, the vitamin E tocotrienol, and derivatives and isomers thereof. As used herein, the term “bixin compound” will be understood to refer to bixin per se and/or its derivatives, including salts and isomers thereof, as well as mixtures thereof including those with the other co-extractives from the annatto seed. The bixin compounds include especially compounds having the basic skeletal structure of norbixin, including its monomethyl ester (bixin), its dimethyl ester (methylbixin), and higher alkyl (e.g., C₂-C₄₀ alkyl) esters, as well as optical isomers thereof and pharmaceutically and dietary acceptable salts thereof.

Bixin is found in nature only in the annatto seed in the form of the 9′-cis isomer. It is converted (hydrolyzed) rapidly in the mammalian body to norbixin in the form of the all-trans isomer. Bixin may be purified from the annatto seed by organic solvent extraction and is available commercially as a color additive for foods, usually as a 1.6 wt % solution in soybean oil (known as annatto color E161b). Bixin and methylbixin may also be prepared synthetically, as described in the literature.

Bixin and its hydrolyzed form, norbixin, are used as coloring agents for human consumption, mainly in cheese (see Collins, “The role of annatto in food colouring,” Food Ingredients and Processing International, 23-27 (February 1992)). For some coloring applications, methylbixin has been commercially prepared by esterification of the carboxylic ester group of the bixin molecule (Zechmelster, et al., “A stereochernical study of methylbixin,” J. Am. Chem. Soc., 66:322330 (1944); Jondiko, et al., “Terpenoids and an apocarotenoid from seeds of B. orellana,” Phytochemistry, 28:3159-3162 (1989)). As a natural color additive to foods, particularly cheeses, bixin is typically added at concentrations of about 1 to 25 mg bixin per kg of food, although higher quantities may be used for some cheeses. Estimates of average daily intake of bixin compounds (usually bixin and norbixin) are of the order of 0.3 to 3 mg per day per person, although some studies report higher values, through the consumption of products such as cheeses, cake decoration, breakfast cereals, ice cream, snacks, margarine, and other foods in which bixin extract is added during food manufacture to provide color. The maximum ADI (acceptable daily intake) established by the Joint FAO/WHO Committee on Food Additives (JECFA, 1982) is 0.065 mg/day/kg body weight, expressed as bixin. For example, for an average human weight of 60 kg, the maximum ADI is 3.9 mg per day. However, ingestion of bixin compounds through food coloring is highly variable and non-systematic. Moreover, bixin compounds are not present at all in many diets, particularly the diets of persons eating only natural foods. Furthermore, bixin compounds and annatto seed extracts are not used in non-human animal foods, not even as coloring agents.

Bixin and its natural derivatives, norbixin and methylbixin, belong to the class of natural compounds called carotenoids. Many important biological functions of carotenoids have been discovered in the past decade (see Krinsky, “Biological properties of carotenoids,” Pure Appl. Chem., 66:1003-1010 (1994)). Major research has been concentrated, however, on the more common carotenoids, such as beta-carotene, lutein, lycopene, and zeaxanthin, and only occasionally has bixin received any attention on the part of the biomedical community. With the discovery that bixin is readily absorbed into the blood stream after human ingestion (Levy, et al., “Bixin and norbixin in human plasma: Discrimination and study of absorption of a single dose of annatto food color,” Analyst, 122:977-980 (1997)), utilization of the biological importance of bixin for mammalian health has become a possibility.

Geranylgeraniol, a 20-carbon isoprenoid alcohol formed in plant systems, was isolated from annatto seeds as such and as the formate, octadecanoate, and famesylacetone forms by Jondiko, et al. and by Craveiro, et al. (“The presence of geranyl-geraniols in B orellana” Quim. Nova 12:297-298 (Chem. Abstr. 112:155274)). Geranylgeraniol bixinates were first reported by Mercadante et al. (“Three minor carotenoids from annatto” Phytochemistry 52:135-139 (1999)). Geranylgeraniol is also formed in animal systems, in which it is synthesized in a complex pathway which also involves the metabolism of cholesterol. The pathway starts with acetyl-coenzyme A, and after three gene-regulated steps and the synthesis of geranylphosphate, a dephosphorylation takes place to form geranylgeraniol (Edwards, et al., “Sterols and isoprenoids: signaling molecules derived from cholesterol biosynthetic pathway” Annu. Rev. Biochem. 68:157-185 (1999)).

Tocotrienols, which are related to the family of tocopherols, were also first reported in annatto seeds by Jodinko. The term vitamin E is now considered to be a generic name describing bioactives of both tocopherol and tocotrienol derivatives. Both consist of a common chromanol head and a side chain at the C-2 position. However, while tocopherol has a saturated phytyl tail, tocotrienol possesses an unsaturated isoprenoid side chain (Sen et al., “Molecular basis of vitamin E action” J Biol. Chem 275(17):13049-13055 (2000)). Tocotrienols are minor plant constituents, especially abundant in palm oil, cereal grains, and rice bran, and can be a considerable source of vitamin E when these products are consumed.

Fresh intact annatto seeds have been used for a long time in traditional medicine of the South American Indians to promote healing of wounds, against skin eruptions and the healing of burns “without a scar,” and have been given internally to subdue diarrhea and asthma (Morton, book review on “Potter's New Cyclopedia of Botanical Drugs and Preparations,” Econ. Botany, 43:280-281 (1989)) and as an antipyretic (Terashima et al. “Studies on aldose reductase inhibitors from natural products:Constituents and aldose reductase-inhibitory effect of C. morifolium, B. orellana and l. batatas,” Chem. Phaz-m. Bull., 39:3346-3347 (1991)). It is not clear if these effects are attributable to bixin or to some of the other components of the annatto seed.

Other botanical parts of the annatto plant also contain physiologically active compounds. Annatto root extract, for example, has been shown to be antisecretory, antispasmodic and hypotensive (Dunham et al., “A preliminary pharmacologic investigation of the roots of B. orellana,” J Am. Pharmac. Assoc., 49:218-219 (1960)), and the aldose reductase inhibitor isoscutellarein has been found in annatto leaf extract (Terashima et al.). Additionally, annatto seed extract given to dogs showed the presence of a hyperglycemic principle (Morrison et al., “Extraction of an hyperglycaemic principle from the annatto, a medicinal plant in the West Indies,” Tropical Geographical Medicine, 43:184-188 (1991)).

Traditionally, the purpose for the use of colors such as bixin (annatto extract) in the food industry has been solely to make a food product look more pleasant and, since “we eat through our eyes,” to make it more palatable. As most natural colors are also potent antioxidants, they also have the function of preserving many nutritionally valuable food ingredients during the shelf life of the product. Over the last few years, more and more evidence has been accumulated that many natural antioxidants, which are useful for the preservation of vitamins and lipids in foods, also produce antioxidant activities in the human body after consumption of these foods. There is mounting evidence of the importance of the antioxidant carotenoids in human health.

Some biological studies in vitro on isolated cell or enzyme systems have shown that bixin or methylbixin shows strong antioxidant activity and lipoxidase inhibition. Bixin has strong physical quenching activity of singlet molecular oxygen and thus the hypothesis has been advanced that it may exert a protective action against some types of cancer (DiMascio et al., “Carotenoids, tocopherols and thiols as biological singlet molecular oxygen quenchers,” Biochem. Soc. Transact., 18:1054-1056 (1990)). However, bixin did not prevent the formation of cancer cells in experimental carcinogenesis with methylcholantrene (Bertram et al., “Diverse carotenoids protect against chemically induced neoplastic transformation,” Carcinogenesis, 12:671-678 (1991)). As a protector against biological membrane oxidation, bixin is a potent inhibitor of lipid peroxidation at the same level of lutein and canthaxanthin and only surpassed by alpha-tocopherol (Zhang et al., “Carotenoids enhance gap junctional communication and inhibit lipid peroxidation in C3H- 1 O-T 1-2 cells: Relationship to their cancer chemopreventive action,” Carcinogenesis, 12:2109-2114 (1991)). Bixin acts as a lipoxydase inhibitor and modulates lipid hydroperoxide formation (Canfield, et al., “Co-oxidations: Significance to carotenoid action in vivo,” Annals New York Academy of Science, 691:192-199 (1993)). Oral administration of bixin significantly reduced the otherwise increased level of lipid peroxides in serum and liver of rats caused by gamma-radiation and can thus be considered a candidate drug for protection against the side-effects in cancer (Thresiamma et al., “Protective effect of curcumin, ellagic acid and bixin on radiation induced lipid peroxidation,” J Exp. Clin. Cancer Res., 14:427-430 (1995); “Protective effect of curcumin, ellagic acid and bixin on radiation induced toxicity,” Indian J Exp. Biol., 34:845-847 (1996) (Chem. Abstr. 125, 241942); and “Protective effect of curcumin, ellagic acid and bixin on radiation induced genotoxicity” J Exp Clin Cancer Res 17(4):431-4 (1998)).

Bixin does not up-regulate Connexin-43 gene expression as some other carotenoids do, but it is active in membrane protection (Zhang et al., “Carotenoids upregulate Connexin-43 gene expression independent of their provitamin A or antioxidant properties,” Cancer Research, 52:5707-5712 (1992)). Although bixin was not specifically included in the study, it has been found that food colors in general enhance immunoglobulin production by rat spleen lymphocytes (Kuramoto et al., “Effect of natural food colorings on immunoglobulin production in vitro by rat spleen lymphocytes,” Bioscience, Biotechnology and Biochemistry, 60:1712-1713 (1996)). Additionally, some interesting spectral changes have been observed during interaction of bixin with respiring rat liver mitochondria (Inada et al., “Spectral changes of bixin upon interaction with respiring rat liver mitochondria,” Arch. Biochem. Biophys., 146:366-367 (1971) and Hirose et al., “Energized state of mitochondria as revealed by the spectral change of bound bixin,” Arch. Biochem. Biophys., 152:36-43 (1972)). Additional reports on the activity of bixin on isolated cells or cell components have appeared in the scientific literature, such as the effect on the respiratory burst of rate peritoneal macrophage (Zhao et al., “Effect of carotenoids on the respiratory burst of rat peritoneal macrophages,” Biochem. Biophys. Acta, 1381:77-88 (1998)) and an induction effect on xenobiotic enzymes of rat lung and kidney (Jewell, et al., “Effect of dietary supplementation with carotenoids on zenobiotic metabolizing enzymes in the liver, lung, kidney and small intestine of the rat,” Brit. J Nutrition, 81:235-242 (1999)).

Mice and rats on high dietary doses of annatto extract or norbixin showed no detectable DNA breakage in liver and kidney (Fernandes et al., “Norbixin ingestion did not induce any detectable DNA breakage in liver and kidney but caused a considerable impairment in plasma glucose levels of rats and mice” J Nutr Biochem 13(7):411-420 (2002)). Further, bixin and norbixin protects DNA in-vitro in chromosomes of cells exposed to radiation, to oxidative damage from metal ions and peroxides, and to cisplatin, the most active cytotoxic agent in the treatment of cancer (Thressiamma, et al. 1998, Kovary et al., “Biochemical behaviour of norbixin during in vitro DNA damage induced by reactive oxygen species”, Br J Nutr 85(4):431-40 (2001), and Silva et al, “Antioxidant action of bixin against cisplatin-induced chromose aberrations and lipid peroxidation in rats”, Pharmacol Res 43(6):551-6 (2001)). Finally, U.S. Pat. Nos. 6,277,378 and 6,316,012 describe topical applications that include bixin compounds which seem to protect the skin from oxidative reactions and carcinogenesis.

It is believed that within the biological defense system against a free radical, the unpaired electron is taken up by the vitamin E, the carotenoids, and vitamin C (ascorbic acid) in a cascade like fashion (Truscott et al., “Carotene:Pro-and antioxidant reaction mechanisms and interactions with Vitamins E and C,” 11th Internat. Symposium on Carotenoids, Leiden (Holland) (August 1996), “The interaction of carotenoids with reactive oxy-species” Biochem Soc Trans 23(2):252S (1995) and Mortensen et al., “The interaction of dietary carotenoids with radical species” Arch Biochem Biophys 385(1):13-9 (2001)). Specifically, vitamin E, after taking up the electron, becomes a transient tocol free radical. The free electron is then transferred to one after the other of the carotenoids through their conjugated double bonds. The first carotenoid of this cycle regenerates the tocol to its original non-free radical state; the second carotenoid regenerates the first carotenoid, and so on. In this process, the excess energy is slowly dissipated as thermal energy during the trip of the electron through the various conjugated double bond systems, and the electron is finally transferred at the lipid/water interface into the aqueous phase where ascorbic acid binds the electron and is excreted from the system, thus finally eliminating the deleterious effects of the free radical.

Geranylgeraniol has been shown to be a very potent inducer of apoptosis (Masuda, et al., “Geranylgeraniol potently induces caspase-3-like activity during apoptosis in human leukemia U937 cells” Biochem Biophys Res Commun 234(3):641-5 (1997); Ohizumi, “Geranylgeraniol is a potent inducer of a poptosis in tumor cells” J Biochem (Tokyo) 117(1):11-3 (1995) and “Induction of apoptosis in various tumor cell lines by geranylgeraniol” Anticancer Res 17(2A):1051-7 (1997)). It caused the selective activation of caspase-3 (Polverino et al, “Selective activation of caspases during apoptotic induction in HL-60 cells. Effects of a tetrapeptide inhibitor.” J Biol Chem 272(11):7013-21 (1997)), a protein that plays a key role in apoptosis (Thornberry et al, “Caspases: enemies within” Science 281(5381):1312-6 (1998)). In U.S. Pat. No. 5,602,184, it is specifically called for in the treatment of prostate cancer. Further, in combination with lovastatin, geranylgeraniol selectively inhibits aberrant (oncogenic) signaling between cells without the cytotoxicity observed when lovastatin is used alone, as described in U.S. Pat. No. 6,083,979 and Ownby et al. (“Farnesol and geranylgeraniol:prevention and reversion of lovastatin-induced effects in NIH3T3 cells”, Lipids 37(2):185-92 (2002)). Additionally, as disclosed in U.S. Pat. No. 5,756,109, geranylgeraniol inhibits esterification of retinol into inactive retinyl esters which may be useful to improve skin desquamation and epidermal differentiation.

It has been found that α-tocotrienol is 40 to 60 times more potent than α-tocotrienol at preventing lipid peroxidation in rat liver microsomes (Sen et al.). Further, Kamat et al. (“Tocotrienols from palm oil as effective inhibitors of protein oxidation and lipid peroxidation in rat liver microsomes”, Mol Cell Biochem 170(1-2):131-7 (1997)) report similar results with a tocotrienol complex in rat mitochondria. Further, it was shown that a combination of 50 ppm tocotrienol plus 50 ppm lovastatin decreases serum cholesterol levels in chickens by 22% and in swine by 35% relative to controls (Qureshi et al., “The combined effect of novel tocotrienols and lovastatin on lipid metabolism in chickens”, Atherosclerosis 156(1):39-47 (2001) and “Synergistic effect of tocotrienol-rich fraction of rice bran and lovastatin on lipid parameters in hypercholesterolemic humans” J Nutr Biochem 12(6):318-329, (2001)). In hypercholesterolemic humans, 100 milligrams of tocotrienols per day for 35 days reduced total cholesterol levels by 20% (Qureshi et al., “Dose dependent suppression of serum cholesterol by tocotrienol-rich fraction (TRF (25)) of rice bran in hypercholesterolemic humans” Atherosclerosis 161(1):199-207 (2002)) and 50 milligrams tocotrienols plus 10 milligrams lovastatin reduced total cholesterol by 25% and significantly increased the HDL to LDL ratio (Qureshi et al., 2002). U.S. Pat. No. 6,441,029 teaches the application of tocotrienols and statins, such as lovastatin, for suppressing tumor growth. In addition to affecting hepatic metabolism, Sen et al. report that tocotrienol protects hippocampal neuronal cells from glutamate-induced death by suppressing activation of the protein c-Src kinase. Further, dietary tocotrienols in pregnant rats are bioavailable to the maternal and fetal brains (Roy et al., “Vitamin E sensitive genes in the developing rat fetal brain: a high density oligonucleotide microarray analysis”, FEBS Lett 530(1-3):17)).

U.S. Pat. No. 6,187,811 describes a method to treat benign prostatic hyperplasia with tocotrienols by themselves and in combinations with a long list of herbs. Further, a method for treatment of neoplastic disease with tocotrienols in combination with limonoids and flavonoids is taught in U.S. Pat. No. 6,239,114.

In the specific case of diabetes, norbixin affects the glycemic levels of rats and mice in surprisingly opposite ways. Specifically, in rats, norbixin significantly induces hyperglycemia ranging from 27% at 8.5 mg dietary norbixin/kg body weight to 53% at 74 mg/kg. In mice norbixin induces up to 22% hypoglycemia at 66 mg norbixin per kilogram body weight (Fernandes et al., “Norbixin ingestion did not induce any detectable DNA breakage in liver and kidney but caused a considerable impairment in plasma glucose levels of rats and mice” J Nutr Biochem 13(7):411-420 (2002)). In vitro tests show that geranylgeraniol upregulates the lipid metabolic target genes in 3T3-L1 adipocytes and HepG2 hepatocytes (Takahashi et al., “Dual action of isoprenols from herbal medicines on both PPARgamma and PPARalpha in 3T3-L1 adipocytes and HepG2 hepatocytes”, FEBS Lett 514(2-3):315-22 (2002)), showing promise for ameliorating lipid metabolic disorders associated with diabetes. Facchini et al. (“Relation between insulin resistance and plasma concentrations of lipid hydroperoxides, carotenoids, and tocopherols”, Am J Clin Nutr 72:776-9 (2000)) found that plasma lipid peroxidation is increased in insulin-resistant individuals well before the development of glucose intolerance and type 2 diabetes. In a double blind crossover study, a tocotrienol complex significantly reduced lipid peroxidation and peroxide levels in 32 non-insulin dependent diabetes mellitus patients (Wan Nazaimoon et al., “Effects of palm olein tocopherol and tocotrienol on lipid peroxidation, lipid profiles and glycemic control in non-insulin diabetes mellitus patients”, Nutrition Research 16:1901-1911 (1996)). In addition, supplementation with vitamin E in humans with type 2 diabetes significantly reduced urinary albumin excretion rate, showing promise as a therapy to prevent end-stage renal disease (Gaede et al. “Double-blind, randomised study of the effect of combined treatment with vitamin C and E on albuminuria in type 2 diabetic patients” Diabet Med 18(9):756-60 (2001)) and decreased plasma levels of C-reactive molecules, a risk factor for myocardial infarction in patients with diabetes (Upritchard et al., “Effect of supplementation with tomato juice, vitamin E, and Vitamin C on LDL oxidation and products of inflammatory activity in type 2 diabetes” Diabetes Care 23(6):733-8 (2000)).

### BRIEF SUMMARY OF THE INVENTION

A method for eliciting a beneficial health effect in a non-human animal is provided. The method comprises adding at least one bixin compound to a feed in an amount effective to increase the bixin concentration in the blood stream of the non-human animal and feeding the feed to the non-human animal at a recommended daily dosage for a sufficient number of days.

A second method for eliciting a beneficial health effect in a human or non-human animal comprises adding at least one bixin compound to a nutritional supplement in an amount effective to increase the bixin concentration in the blood supply of the human or non-human animal and feeding the nutritional supplement to the human or non-human animal at a unit dosage for a sufficient number of days.

A method for eliciting a therapeutic health effect in a human or non-human animal comprises adding at least one bixin compound to a pharmaceutical composition in an amount effective to increase the bixin concentration in the blood stream of the human or non-human animal and feeding the composition to the human or non-human animal at a unit dosage for a sufficient number of days.

Finally, a nutritional supplement for a human or non-human animal comprises at least one bixin compound in an oral pharmaceutical or neutraceutical dosage for administration to the subject, wherein the dosage form provides a dosage of at least about 6 mg per day of the at least one bixin compound to the subject.

### DETAILED DESCRIPTION OF THE INVENTION

There is increasing awareness of the importance of controlling metabolic production of free radicals in mammalian bodies, especially amongst companion animals, for good health. This may be accomplished by consumption of foods from the plant kingdom that are rich in antioxidants, such as fruits and vegetables. In the case of traditionally carnivorous animals, such as dogs and cats, it is theorized that their intake of plant antioxidants is accomplished by consuming herbivorous prey that contain plant-derived antioxidants in their flesh and blood. In the case of companion animals in modern urban societies, however, the consumption of manufactured pet foods has reduced the dietary intake of plant-derived antioxidants of those naturally available “in the wild.” Consequently, there exists a need to supplement the diet of companion animals with these antioxidants in order to attain a natural balance for good health.

Bixin is one of the most powerful carotenoid antioxidants. Its conjugated double bond system is ideally suited for scavenging free radicals. It is also unique among the natural carotenoids in that it is a very polar molecule due to the presence of two carbonyl groups. As such, the bixin molecule may locate at the lipid/water interface of the cell membrane which may give it an especially important potential action in preserving health of mammals, both human and non-human.

When other carotenoids are also supplemented in conjunction with bixin, the antioxidant action of bixin is enhanced as the step-wise energy dissipation ladder is strengthened by the additional carotenoids. There is ample evidence of the activity of lutein, beta-carotene, alpha-carotene, zeaxanthin, astaxanthin, and cryptoxanthin in the free-radical scavenging system.

Accordingly, according to the present invention, beneficial health effects in animals, both human and non-human, have been shown to be elicited by daily supplementation of food with bixin and bixin-derivatives. Such supplementation may enhance the activity of the immune system, making these animals less prone to gastrointestinal infections and infections of the more systemic kind. When such animals are companion animals, such as cats and dogs, this may be of great importance to the general well being of the animals (and the satisfaction of their owners). The beneficial health effects are also desirable to farmed animals, such as layer hens, broiler chickens, and pigs.

Specifically, the beneficial health effect may be elicited by adding at least one bixin compound to a food or feed product, a nutritional product or supplement, or a pharmaceutical product or supplement, in an amount effective to increase the bixin concentration in the blood supply, and feeding the feed, product or supplement to the human or non-human animal at a recommended daily dosage (unit dosage) for a sufficient number of days.

The bixin compound may be bixin, norbixin, methylbixin, higher alkyl esters and diesters of norbixin, isomers thereof, and/or extracts of the annatto (Bixa orellana) seed. The beneficial health effect may be further enhanced by adding to the food product, feed, or nutritional supplement, in addition to the bixin compound(s), at least one carotenoid compound selected from the group consisting of lutein, zeaxanthin, beta-carotene, alpha-carotene, astaxanthin, lycopene, cryptoxanthin and the esters, diesters and isomers thereof, or an extract of marigold, corn, spinach, alfalfa, algae, bacteria, tomato, or citrus which contains at least one of these carotenoid compounds. Preferably, the carotenoid compound is lutein, lutein ester, lutein diester, and/or extract of marigold.

The recommended daily amount or unit dosage of the food product, feed, or nutritional supplement and the number of days such a product or supplement may be ingested in order to elicit the beneficial health effect are determined by a number of factors which affect the food intake of the animal. These factors include the particular species of animal, its age, body weight, and activity level, as well as the animal's particular living conditions, including the season and the outside temperature and climate. For example, an active animal will consume more than one that is passive, and an animal living on a farm will be more active than one living in a city. Further, animals living on a farm, for example, will consume different amounts of food depending on the time of year.

When the bixin compound(s) are added to a feed or food product, the effective amount is preferably at least about 1 mg bixin per kg of the feed, more preferably at least about 10 mg per kg, and most preferably at least about 50 g per kg. In a nutritional product or supplement, the effective amount of bixin compound(s) is preferably at least about 250 micrograms (μg) per unit dosage, more preferably at least about 1 mg per unit dosage, and most preferably at least about 10 g per unit dosage. However, as discussed above, various factors affecting the food consumption of the animal will affect the amount of bixin that is effective. The unit dosage of the nutritional supplement may be administered parenterally or orally as a tablet, capsule, or liquid.

This invention also relates to a method for administering at least one bixin compound in a pharmaceutical composition to elicit a therapeutic health effect in a human or non-human animal by increasing the bixin concentration in the blood stream of the animal or human, and feeding the composition to the animal or human at a unit dosage for a sufficient number of days. In addition to a more frequent supplementation of bixin, such a method may be desirable for mammals under particular stress situations in order to boost the function of their immune systems. This method is particularly suited to cats and dogs, but may be applicable to any animal or human.

The effective amount of the bixin compound(s), which may be any as previously described, in such a pharmaceutical composition is preferably at least about 400 μg per unit dosage, more preferably at least about 30 mg, and most preferably at least about 2 g per dosage. However, as explained above, various conditions will affect the amount of bixin that is effective, including the species of animal, its age, body weight, activity level, and living conditions.

As previously described, the recommended daily amount or unit dosage of the pharmaceutical composition and the number of days such a product or supplement may be ingested in order to elicit the therapeutic health effect are determined by a number of factors which affect the food intake of the animal or human. The therapeutic health effect may be further enhanced by the addition of at least one carotenoid compound to the pharmaceutical composition, such as lutein, zeaxanthin, beta-carotene, alpha-carotene, astaxanthin, lycopene, cryptoxanthin and the esters, diesters and isomers thereof, or at least one extract of marigold, corn, spinach, alfalfa, algae, bacteria, tomato, or citrus containing these carotenoid compounds. Preferably, the carotenoid compound is lutein, lutein ester, lutein diester and/or extract of marigold.

The therapeutic health effects which may be elicited include, but are not limited to enhancing activity of the immune system, reducing systemic infection, improving symptoms of diabetes, inhibiting cancer development, inhibiting atherosclerosis and/or heart disease, lowering LDL cholesterol, reducing inflammation of an internal organ, and protecting neuron cells from being killed due to stroke, inflammation, or neurodegenerative disease.

It has been found that the ingestion of bixin compounds by humans at dosage forms above those traditionally used to simply provide a color effect in food or other ingestibles increases the antioxidant potential of the plasma and, as such, aids the other nutritional antioxidants in providing anti-tumor activity, protection against atherosclerosis, as well as aiding the entire range of antioxidant activity necessary for human health. Accordingly, a nutritional supplement for a human or non-human animal is provided by the invention. The supplement comprises at least one bixin compound, which may be any as previously described, in an oral pharmaceutical or neutraceutical dosage for administration to the subject.

The dosage form provides a dosage of the bixin compound(s) of at least about 3 mg per day (based on an average subject of 60 kg body weight, i.e., at least about 0.05 mg per kg body weight per day), preferably at least 6 mg per day up to about 50 mg per day, and more preferably about 10 to 30 mg per day. In view of the rapid clearance rate of bixin from the blood stream, it is preferred to administer the bixin compound in two or more doses per day. While the above amounts are preferred, it is believed that persons who do not ingest bixin compounds at all from their normal diets or do not ingest bixin compounds in significant or regular amounts may benefit by the administration of as little as one or 2 mg per day on a regular basis.

The bixin compound(s) in the nutritional supplement may be administered orally or parenterally in usual nutritional and/or pharmaceutical forms, such as pills, capsules, injectable solutions, and the like, containing an amount of bixin compound(s) to deliver a dosage of at least 1 mg bixin compound, preferably greater than 3 mg bixin compound, and more preferably at least 6 mg of bixin compound. Preferred for convenience are oral forms, such as soybean oil solutions injected into capsules, and microbeads or compressed tablets.

The compositions further comprise acceptable carriers or excipients which are not critical. Such acceptable carriers and excipients are well known to those skilled in the art of nutritional supplements and pharmaceutical compositions. These include, but are not limited to, solid carriers such as carbonates and silicates, and liquid excipients such as water, alcohol, soybean oil, vegetable oil, and glycol.

The bixin compound may also be added directly to foods, to be administered as a “fortified food.” Here, the bixin compound(s) should be present at a level of at least 50 mg per kg food, and preferably at least 80 mg per kg food. According to the invention, beneficial health effects for the consumer may be achieved when the bixin compound level in the food is increased to 80 mg/kg or above, as it is at these levels that actual therapeutic effects of the bixin compound may be achieved. With an increased level of bixin compound, the food becomes a “fortified food” from which the benefit of the bixin compound can be obtained by the consumer.

Bixin is unique among the natural carotenoids, in that it is a very polar molecule from the presence of two carboxyl groups. While not wishing to be bound by any particular theory, it is believed that the bixin compound molecule may locate at the lipid/water interfaces of the cell membrane, which gives it an especially important potential action in preserving animal health, both human and non-human. Preliminary results show that bixin compounds have important beneficial actions on the human body. Specifically, bixin compounds after ingestion regulate gene expression in lymphocytes and may control T-cell proliferation, differentiation, and apoptosis. This suggests that bixin and its derivatives may inhibit cancer developments as an immunological adjuvant producing large amounts of IgM antibodies and some IgG antibodies. Furthermore, preliminary results demonstrate that the oxidation of the LDL and VLDL fractions of cholesterol is effectively inhibited by bixin compounds, thus aiding in the prevention of atherosclerosis and heart disease.

Accordingly, the purified bixin compounds may be taken orally as a prescription drug by patients suffering from heart disease, atherosclerosis, or other diseases caused directly or indirectly by free-radical molecules formed in the blood stream by excessive biological oxidation reactions. They can also be used for this purpose via parenteral administration.

Bixin compounds may also be used as a nutritional supplement in forms such as vitamin pills or food additives by anyone who wants to reduce his or her risk of diseases caused by free radicals or to strengthen the immune system.

The position of bixin compounds in the metabolic cycle has not yet been clearly established. However, research results show that bixin compounds fit prominently into the scheme of protecting the animal body against the harmful effects of free radicals. Free radical scavenging activity is related to the number of conjugated double bonds in the carotenoid molecule. Bixin is in this group of molecules and is one of the most powerful carotenoid antioxidants. Its conjugated double bond system is ideally suited for scavenging free radicals. After consumption, it is well absorbed into the blood stream. Therefore, bixin compounds are important new nutritional supplements to improve human and non-human animal health.

The invention will be further understood in conjunction with the following, non-limiting examples.

### EXAMPLE 1

Two groups of human volunteers (3 individuals in the test group and 5 in the control group) were exposed to high oxidative stress conditions by breathing contaminated air of a high ozone level and a high concentration of smoke over a period of four to five days for one half hour per day. Both groups were given the same diets. Namely, they consumed their normal daily diets but avoided foods rich in carotenoids, particularly foods containing annatto (as a colorant), from two weeks before the exposure until two weeks after the exposure. The test group ingested a daily dose of 20 milligrams of bixin administered in one capsule of 10 milligrams after breakfast and another capsule of 10 milligrams during dinner, while the control group did not receive any bixin supplementation.

During the period of bixin supplementation, the serum level of bixin and norbixin was zero in the control group, while the serum levels of bixin and norbixin in the individuals of the test group reached levels of 12 and 60 micrograms/liter, respectively.

During and after the exposure to the high oxidative stress conditions, the oxidative stress load in the blood of the control group was high, as measured by a modified “TBAR” (thiobarbituric acid-reactive substances) test, whereas in the test group that received the bixin supplementation, the oxidative stress level of the blood remained normal. The same relation was found in the determination of free radical content of the blood.

These results demonstrate the protective effect obtained from bixin supplementation against the harmful action of the free radicals formed by the high oxidative stress to which the subjects were exposed. It is thus reasonable to predict that subjects receiving the protection of administered bixin will in the long term have a lower risk of developing heart disease and cancer. Moreover, it would be reasonable to assume that their immune systems will remain undiminished and, in fact, would be enhanced, based on clinical in vitro trials with isolated splenocytes.

### EXAMPLE 2

Female BALB/c mice and the WAZ-2T(-SA) mammary tumor cell line were used to determine the efficacy of dietary bixin in inhibiting mammary tumor development. Six mice per treatment were fed a semi synthetic AlN-93M diet containing 0, 0.006 or 0.06% bixin. After 14 days, all mice were inoculated with 2500 tumor cells (a cell load which typically produces a 65% tumor incidence in untreated controls). Tumor growth was measured daily for 50 days, at which time blood, liver, spleen, and tumors were removed and evaluated. Plasma bixin and norbixin levels showed dose-dependent increases which peaked at 400 micrograms norbixin and 30 micrograms bixin/L. Mammary tumor incidence and tumor growth rate were lowest in the 0.06% dietary bixin level and increased in a dose dependent manner. Final tumor weight was 0.3 grams at 0.06% dietary bixin, 0.7 grams at 0.006% and 1.1 grams for the control without dietary bixin. Tumor latency (number of days post inoculation when mammary tumors were first palpable) was 45 days at 0.06% dietary bixin level, 38 days at 0.006%, and 30 days for the control. Results were determined to be statistically significant at the P<0.05 level. Analytical work on isolated splenocytes cultured in the presence of concanavalin-A determined dose dependent effects on mRNA expression levels of PIM-1, p-53, BAX, and BCL-2. It may thus be concluded that the dietary bixin protected the mice from mammary tumor development by triggering genes of immune related systems rather than by an antioxidant action at the cellular level.

### EXAMPLE 3

Two type 2 diabetic patients in general good health taking sulfonylurea but no other medication or insulin were studied. The patients, aged 32 and 45 and having body mass indexes of 29 and 33 kg/m², took 20 milligrams annatto extract orally, daily at dinner, for 12 weeks as a suspension in oil. Key parameters were measured monthly, in which subjects ingested 75 grams of glucose in 300 mL flavored water and plasma glucose and insulin levels were measured every 15 minutes for 2 hours. Fasting plasma glucose levels before treatment were 174 and 184 mg/dl. Further, monthly measurements showed a significant falling trend which after 12 weeks of treatment ended with levels of 125 and 128 mg/dl. Plasma HbAIC significantly decreased monthly over the course of the treatment from 8.6% and 8.9% before treatment down to 6.8% and 6.8% after treatment. No changes were observed in fasting or glucose-stimulated insulin/C-peptide concentrations. From experience in similar populations as these two patients, the results suggest that the annatto extract treatment decreased lasting and postprandial plasma glucose levels by improving hepatic and peripheral (muscle) tissue sensitivity to insulin.

It has thus been found, according to the present invention, that bixin compounds and annatto extracts, especially in dosage amounts above the levels normally used for coloring purposes, exert a profound beneficial effect on the health of humans and non-human animals. Bixin compounds may thus be used as nutritional supplements, food fortifiers, and pharmaceuticals in the form of tablets, capsules, liquid preparations and the like to improve the health of animals by the stimulation of immune systems, the amelioration of the symptoms associated with diabetes, the reduction of inflammatory processes of internal organs, and the protection from death of neuronal cells. Such compounds may thus be used to inhibit cancer development by regulating gene expression in lymphocytes and controlling T-cell proliferation, differentiation and apoptosis, to prevent or inhibit atherosclerosis and heart disease by inhibiting the oxidation of LDL and VLDL fractions of cholesterol, and generally strengthening the immune system and reducing the risk of diseases caused directly or indirectly by excessive biological oxidation reactions by free radical molecules formed in the blood stream.

Furthermore, the nutritional supplementation of humans and non-human animals with bixin compounds and other annatto seed extractives has protective and therapeutic health effects. Although a method to separate the components of the annatto seed extract by distillation in order to use these components individually as supplements or as building blocks for chemical synthesis is described in U.S. Pat. No. 6,350,453, according to the present invention the bixin compounds and other annatto seed extractives seem to cause a much more significant synergistic protective and therapeutic health effect when ingested together than when the components are administered individually.

Finally, the present invention provides for the dietary inclusion of these compounds which stimulates beneficial physiological functions and will allow for the manufacture of improved pet food formulae, for use in veterinary diets, which are therapeutic to address specific ailments.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for eliciting a beneficial health effect in a non-human animal comprising adding at least one bixin compound to a feed for the non-human animal in an amount effective to increase a bixin concentration in a blood stream of the non-human animal, and feeding the feed to the non-human animal at a recommended daily dosage for a sufficient number of days.

2. The method according to claim 1, wherein the at least one bixin compound is selected from the group consisting of bixin, norbixin, methylbixin, higher alkyl esters and diesters of norbixin, isomers thereof and extracts of the annatto (Bixa orellana) seed.

3. The method according to claim 1, wherein the beneficial health effect is enhanced by adding to the feed at least one carotenoid compound selected from the group consisting of lutein, zeaxanthin, beta-carotene, alpha-carotene, astaxanthin, lycopene, cryptoxanthin and the esters, diesters and isomers thereof, or at least one extract selected from the group consisting of extracts of marigold, corn, spinach, alfalfa, algae, bacteria, tomato, and citrus, wherein the at least one extract contains at least one of the carotenoid compounds.

4. The method according to claim 3, wherein the at least one carotenoid compound is selected from the group consisting of lutein, lutein esters, lutein diesters, and extracts of marigold.

5. The method according to claim 1, wherein the non-human animal is a companion animal.

6. The method according to claim 5, wherein the companion animal is selected from the group consisting of a dog and a cat.

7. The method according to claim 1, wherein the non-human animal is a farmed animal selected from the group consisting of a layer hen, a broiler chicken, and a pig.

8. The method according to claim 1, wherein the effective amount of the at least one bixin compound is at least about 1 mg per kg of the feed.

9. The method according to claim 8, wherein the effective amount of the at least one bixin compound is at least about 10 mg per kg of the feed.

10. The method according to claim 9, wherein the effective amount of the at least one bixin compound is at least about 50 g per kg of the feed.

11. A method for eliciting a beneficial health effect in a human or non-human animal comprising adding at least one bixin compound to a nutritional supplement in an amount effective to increase a bixin concentration in a blood supply of the human or non-human animal, and feeding the nutritional supplement to the animal at a unit dosage for a sufficient number of days.

12. The method according to claim 11, wherein the at least one bixin compound is selected from the group consisting of bixin, norbixin, methylbixin, higher alkyl esters and diesters of norbixin, isomers thereof, and extracts of the annatto (Bixa orellana) seed.

13. The method according to claim 11, wherein the beneficial health effect is enhanced by adding to the nutritional supplement at least one carotenoid compound selected from the group consisting of lutein, zeaxanthin, beta-carotene, alpha-carotene, astaxanthin, lycopene, cryptoxanthin and the esters, diesters and isomers thereof or at least one extract selected from the group consisting of extracts of marigold, corn, spinach, alfalfa, algae, bacteria, tomato, and citrus, wherein the at least one extract contains at least one of the carotenoid compounds.

14. The method according to claim 13, wherein the at least one carotenoid compound is selected from the group consisting of lutein, lutein esters, lutein diesters and extracts of marigold.

15. The method according to claim 11, wherein the effective amount of the at least one bixin compound in the unit dosage of the nutritional supplement is at least about 250 μg.

16. The method according to claim 15, wherein the effective amount of the at least one bixin compound in the unit dosage of the nutritional supplement is at least about 1 mg.

17. The method according to claim 16, wherein the effective amount of the at least one bixin compound in the unit dosage of the nutritional supplement is at least about 10 g.

18. The method according to claim 11, wherein the unit dosage of the nutritional supplement is administered parenterally or orally as a tablet, a capsule, or a liquid.

19. A method for eliciting a therapeutic health effect in a human or non-human animal comprising adding at least one bixin compound to a pharmaceutical composition in an amount effective to increase a bixin concentration in a blood supply of the human or non-human animal, and feeding the pharmaceutical composition to the animal at a unit dosage for a sufficient number of days.

20. The method according to claim 19, wherein the at least one bixin compound is selected from the group consisting of bixin, norbixin, methylbixin, higher alkyl esters and diesters of norbixin, isomers thereof, and extracts of the annatto (Bixa orellana) seed.

21. The method according to claim 19, wherein the therapeutic health effect is enhanced by adding to the pharmaceutical composition at least one carotenoid compound selected from the group consisting of lutein, zeaxanthin, beta-carotene, alpha-carotene, astaxanthin, lycopene, cryptoxanthin and the esters, diesters and isomers thereof or at least one extract selected from the group consisting of extracts of marigolds, corn, spinach, alfalfa, algae, bacteria, tomato, and citrus, wherein the at least one extract contains at least one of the carotenoid compounds.

22. The method according to claim 21, wherein the at least one carotenoid compound is selected from the group consisting of lutein, lutein esters, lutein diesters and extracts of marigold.

23. The method according to claim 19, wherein the effective amount of the at least one bixin compound in the unit dosage of the pharmaceutical composition is at least about 400 μg.

24. The method according to claim 23, wherein the effective amount of the at least one bixin compound in the unit dosage of the pharmaceutical composition is at least about 30 mg.

25. The method according to claim 24, wherein the effective amount of the at least one bixin compound in the unit dosage of the pharmaceutical composition is at least about 2 g.

26. The method according to claim 19, wherein the therapeutic health effect is selected from the group consisting of enhancing an activity of an immune system, reducing a systemic infection, improving symptoms of diabetes, inhibiting cancer development, inhibiting atherosclerosis and/or heart disease, lowering LDL cholesterol, reducing an inflammation of an internal organ, and protecting neuron cells from being killed due to a stroke, an inflammation, or a neurodegenerative disease.

27. The method according to claim 19, wherein the unit dosage of the pharmaceutical composition is administered parenterally or orally as a tablet, a capsule, or a liquid.

28. A nutritional supplement for a human or non-human animal comprising at least one bixin compound in an oral pharmaceutical or neutraceutical dosage for administration to the subject, wherein the dosage form provides a dosage of at least about 6 mg per day of the at least one bixin compound to the subject.

29. The nutritional supplement according to claim 28, wherein the at least one bixin compound is selected from the group consisting of bixin, norbixin, methylbixin, higher alkyl esters and diesters of norbixin, isomers thereof and extracts of the annatto (Bixa orellana) seed.

30. The composition according to claim 28 wherein the composition is administered parenterally or orally as a tablet, a capsule or a liquid.
